(19) United States
(12) Patent Application Publication
(10) Pub. No.: US 2012/0184626 A1
(43) Pub. Date: Jul. 19, 2012

(54) GERMICIDAL TOPICAL COMPOSITIONS

(75) Inventor: Sandra Judith Guerra-Vega, Montvale, NJ (US)

(73) Assignee: Reckitt Benckiser LLC, Parsippany, NJ (US)

(21) Appl. No.: 13/498,443

(22) PCT Filed: Jun. 15, 2011
(86) PCT No.: PCT/GB2011/051117

§ 371 Date: Mar. 27, 2012

(30) Foreign Application Priority Data
Jun. 18, 2010 (GB) 1010256.4

(51) Int. Cl. A61K 31/045; A61P 31/04

(52) U.S. Cl. 514/724

(57) ABSTRACT

A topical germicidal composition comprising: 50-85% wt. of an alcohol constituent comprising one or more C1-C4 monohydric alcohols; 0.01-5% wt. of a film forming constituent based-on one or more celluloses or cellulose derivatives, 0.01-25% wt. of a humectant, preferably glycerine; 0.01-5% wt. of an opacifier constituent; optionally but preferably a Polyquaternium-type polymer or material; optionally one or more further constituents for improving the aesthetic or other technical characteristics of the invention, with the balance-of the composition, up to about 30% wt. of water, wherein the composition is characterized that it is flowable and preferably also exhibits an initial viscosity (“as mixed”) of at least about 10 cPs measured at 25° C., and subsequent to being stored at elevated temperatures and/or extended time intervals are retained as a single phase composition and do not split or separate into two or more phases, and further, the compositions provide a topical germicidal benefit when applied to the skin or parts of the body.

The present invention relates to germicidal topical compositions which have a high alcohol content, and which provide a germicidal benefit to dermal surfaces upon which the compositions are applied.

Topical compositions, per se, are well-known in the cosmetic, dermatological as well as in the pharmaceutical fields. Most topical compositions are intended to provide at least one but generally provide multiple or more specific benefits after being applied to the human skin. For example, personal care compositions which are primarily intended to be soaps for general cleaning of the human skin such as hand soaps or body wash soaps are well known in the fields of cosmetics and personal care products. While providing a primary cleaning benefit, such personal care compositions frequently also provide ancillary benefits such as moisturizing and nourishing the skin. Such personal care compositions which provide a good general cleaning benefit are usually based on one or more anionic soaps or anionic surfactants which are recognized to provide good cleaning and good foaming. However, such compositions typically provide only limited germicidal benefits.

Also known to the art, are topical compositions which are primarily directed to provide a germicidal benefit to the epidermis or other body part when applied thereto. Such typically take the form at viscous eels and are often lamely composed of an alcohol, usually ethanol, with Ember constituents, e.g., thickeners. While alien technically effective to provide a germicidal benefit, such compositions are also not without shortcomings, including in some cases an unpleasant skin feel and in other cases, an undesired drying effect to the skin.

One such composition which has been proposed in the art is a “Formulation number: US-371-666-145-8” (ex. Cognis Corp., Ambler Pa.) which discloses a “Hand Santizer with Aloe” which includes the following constituents:

| Constituent: | % wt. |
|---|---|
| water | 25 |
| glycerin | 3 |
| ethanol | 62 |
| polymer based on Polyquaternium-37 and dicaprylyl | 2 |
| carbonate and lauryl glycoside (Cosmedia ® | |
| Triple C) | |
| propylheptyl caprylate (Cetiol ® SenSoft) | 2 |
| sodium hydroxide (10%) | -minor- |
| water and Cassia Angustifolia seed polysaccharide | 5 |
| Aloe Primsponge | 1 |

The foregoing composition is stated to exhibit a pH of 4.5, and a viscosity of 45,000 cPs as measured using a Brookfield RVF viscometer, at 23° C., speed T-E, 5 rpm, Helipath. The foregoing a composition however is not immune from shortcomings, and may be thriller improved.

Further topical antimicrobial compositions are known from US 2009/0226497 which describe antimicrobial skin sanitizing compositions comprising a high proportion of an alcohol, a cationic compound, e.g., a skin conditioning cationic compound such as one or more polyquaternium compounds, and one of a selected group of thickeners, e.g., PEG-150 stearate. PEG-150 distearate, PEG-175 diisostearate, polyglyceryl-10 behenate/eicosadioate, disteareth-100 IPDI polyacrylamidomethylpropane sulfonic acid, butylated PVP, and combinations thereof (see para. [0027]). The document notes (at para. [0025]) that thickening systems including those based on cellulosic polymers, starches, acrylates, and/or acrylate based polymers are to be avoided in compositions having a high alcohol content and wherein cationic compounds are also present. These selected group of thickeners are identified as being compatible with the cationic, compounds present in the composition as not precipitating or coascervating (see para. [0026]).

It is to these shortcomings as well as further shortcomings in the art to which the current invention is directed.

In a first aspect of the invention there are provided topical germicidal compositions for application to the epidermis, e.g., hands, arms, legs, lace, scalp as well as other body areas.

According to a second aspect of the invention is provided a method for the manufacture or production of improved topical germicidal composition as set forth herein.

Broadly stated, in a third aspect of the invention there are provided topical germicidal compositions for application to the epidermis, e.g., hands, arms, legs, face, scalp as well as other body areas. In certain preferred embodiments, the topical germicidal compositions are those which are flowable and exhibit an initial viscosity of at in the range of 10-100,000 cP at 25° C. as measured using conventional quantitative methods. These topical germicidal compositions comprise (in preferred embodiments consists of, or consists essentially of):

about 50-85% wt., preferably about 55-70% wt, of an alcohol constituent comprising one or more C₁-C₄ monohydric alcohols;

about 0.01-5% wt, preferably about 0.01-1% wt. of a film forming constituent based on one or more celluloses or cellulose derivatives, such as hydroxypropylmethylcellulose:

about 0.01-25% wt. of a humectant, preferably glycerine:

about 0.01-5% wt, of an opacifier constituent, preferably an anionic opacifier constituent;

optionally but preferably a Polyquaternium-type polymer or material;

optionally one or more further constituents for improving the aesthetic or other technical characteristics of the invention,

with the balance of the composition, up to about 30% wt. of water, wherein the composition is characterized that it is flowable and preferably also exhibits an initial viscosity (“as mixed”) of at least about 10 cPs measured at 25° C., and subsequent to being stored at elevated temperatures and/or extended time intervals are retained as a single phase composition and do not split or separate into two or more phases, and further, the compositions provide a topical germicidal benefit when applied to the skin or parts of the body.

According to a fourth aspect of the invention there is provided an improved method for the treatment of the skin (epidermis) as well as other body surface including the hair which method includes the application of a cleaning and/or germicidally effective amount of the topical composition described herein in order to provide an effective cleaning and/or germicidal benefit.

In a fifth aspect, the present invention provides a topical germicidal composition according to the any of the prior aspects of the invention, characterized in that the said composition is effective against one or more preferably at least two or more of the following microorganisms: B. cepacia, E. coli, S. aureus, S. marcenscens, S. pyogenes, S. epidermidis. E. faecalis, K. pneumoniae, P. aeruginosa, E. hirae, S. pneumoniae, C. albicans, S. enterica, and methicillin resistant Staphylococcus aureus (“MRSA”).

According to a sixth aspect, there is provided a the topical germicidal compositions as described herein which may be provided in a variety of vendible product forms, e.g., viscous flowable forms, such as gels, creams or pastes as well as readily flowable forms adapted to be poured from a bottle or flask, or more flowable forms suitable to be dispensed from such a bottle, flask or other reservoir via a nozzle or a pump, e.g., a manually operable pump or a manually operable trigger spray.

These and further aspects of the invention are provided as described within this specification.

The primary constituent of the topical germicidal compositions is an alcohol constituent, comprising one or more C₁-C₄ monohydric alcohols, e.g., one or more alcohols selected from methanol, ethanol, n-propanol, isopropanol, and all isomers of butanol. Isopropanol, although often used on the skin, is less desirable for use in the present invention because of severe defatting tendency. Its detailing tendency may, however, be compensated for by adding sufficient emollient ingredient if desired to offset this tendency. Preferred alcohols according to the present invention are however ethanol and n-propanol, and especially preferably ethanol to the exclusion of further C₁-C₄ monohydric alcohols. In the present invention, when more than one alcohol is used, the alcohols are mixed at a concentration, that is peak for their activity. Ethanol is included for its reduced defining activity and for activity against viruses, especially the lipophilic group; while the inclusion of n-propanol enhances the contribution of the alcohol constituent to the overall germicidal efficacy of the topical germicidal compositions of which they form a part. In certain preferred embodiments the alcohol constituent comprises at least 50% wt., or on order of increasing preference) at least 55% wt., 60% wt., 65% wt., 70% wt., 75% wt., 80% wt., 85% wt., 95% wt., and especially preferably comprises at least 100% wt, ethanol. The alcohol constituent itself comprises at least 50% wt., preferably comprises at least 55% wt., still more preferably comprises at least 60% wt. of the topical germicidal compositions of which a forms a part. Concurrently the alcohol constituent desirably comprises not more than 85% wt., preferably not more than 80% wt., still more preferably not more than and especially preferably comprises not more than 70% wt. of the topical germicidal compositions. Particularly preferred amounts of the alcohol constituent and the identity thereof are disclosed in one or more of the following examples.

The compositions of the invention necessarily also include a film forming constituent based on cellulose or one or more cellulose derivatives. Such film forming constituents are per se, known to the art and exemplary useful cellulose derivatives useful as a film forming constituent include methyl cellulose ethyl cellulose, hydroxymethyl cellulose hydroxy ethyl cellulose, hydroxy propyl cellulose, carboxy methyl cellulose, carboxy methyl hydroxyethyl cellulose, hydroxypropyl cellulose, hydroxy propyl methyl cellulose, ethylhydroxyethyl cellulose and ethyl hydroxy ethyl cellulose. Of the foregoing hydroxypropyl methyl cellulose is particularly preferred for use in preferred compositions of the invention.

The film forming constituent based on cellulose or one or more cellulose derivatives is advantageously present in an amount of from about 0.01-5% wt., in order of increasing preference comprises at least about 0.01% wt., 0.05% wt., 0.1% wt., 25% wt., 0.15% wt., 0.175% wt., 0.2% wt., of the composition of which it forms a part, and concurrently, the film firming constituent based on cellulose or one or more cellulose derivatives is advantageously present in an amount not in excess of about 5% wt., 4% wt., 3.5% wt., 3% wt., 2.5% wt., 1.75% wt., 1.5% wt., 1.25 wt., and 1% wt., based on the total weight of the composition of which it forms a part. Particularly preferred amounts of the film forming constituent and the identity thereof are disclosed in one or more of the following examples.

The inventor has surprisingly found that a very high increase in the viscosity of the compositions may be attained on use of even small amounts of the film forming constituent based on cellulose or one or more cellulose derivatives in the compositions which contain a major proportion of the alcohol constituent, and, that in preferred embodiments the inventive compositions remain storage stable even under harsh storage conditions.

The topical germicidal compositions comprise one or more humectants, including polyhydric alcohols including polyalkylene glycols as well as alkylene polyols and their derivatives, inter alia, including propylene glycol, dipropylene glycol, polypropylene glycol, polyethylene glycol and derivatives thereof, sorbitol, hydroxypropyl sorbitol, etythritol, threitol, pentaerythritol xylitol, mannitol, hexylene glycol, butylene glycol (e.g., 1,3-butylene glycol), hexane triol (e.g., 1,2,6-hexanetriol), glycerine, ethoxylated glycerine and propoxylated glycerine. Further useful humectants include sodium 2-pyrrolidone-5-carboxylate, guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; and panthenol. Still further humectants include polyols e.g., linear and branched chain alkyl polyhydroxyl compounds such as, propylene glycol, polyethylene glycol, glycerine and sorbitol. Exemplary hydrocarbons which may also serve as humectants are those having hydrocarbon chains anywhere from 12 to 30 carbon atoms, particularly, mineral oil petroleum jelly, squalene and isoparaffins.

The humectants may be used singly or two or more humectants may be included in topical germicidal compositions of the invention. In preferred embodiments, one or more humectants may be included in effective amounts, advantageously from 0.01-25% wt., preferably horn 5-15% wt, based on the total weight of the composition of which it forms a part. In particularly preferred embodiments, a humectant is necessarily present in an amount of from 5-12.5% wt., and a particularly preferred humectant is selected from polyhydroxy alcohols, such as glycerine and/or alkoxlated polyhydroxy alcohols, such as ethoxylated glycerine and propoxylated glycerine. Of the foregoing, glycerine either used singly or in conjunction with aloe vera is a particularly preferred humectant.

The inventive compositions also an opacifier constituent. Such are materials which are typically emulsions, dispersions or suspensions of a water insoluble polymer or copolymer in a carrier. Such may also be referred to as latexes. The carrier may be aqueous, an aqueous/organic solvent mixture or organic solvent. The opacifier constituent may based on a homopolymer, or on copolymer. It is contemplated that the copolymer comprises two or more different monomers which are joined neither a block or random arrangement of the two or more different monomers.

Exemplary copolymers suitable for the pacifier include those formed from styrene, alpha-methylstyrene, divinylbenzene, acrylic acid, methacrylic acid, C₁-C₂₀ esters of acrylic acid or methacrylic acid, acrylamide, methacrylamide, maleic acid, vinyl acetate, crotonic acid, vinyl neodecanoate and butenoic acid. Examples of carboxylate type copolymers are the styrene/alkyl acrylate and partially esterified polyacrylic and polymethacrylic salts and free acid forms. Among the foregoing materials are poly(butyl methacrylate), poly(methyl acrylate), poly(methyl methacrylate), poly(acrylic acid/C₁-C₂₀ alkyl acrylate) and poly(meth acrylic acid/C₁-C₂₀ alkyl methacrylate). These copolymers may be prepared by polymerization of the respective monomers by traditional oil-in-water or water-in-oil emulsion polymerization techniques. Alternatively, a pseudo latex useful as an opacifier constituent may be prepared by esterification of preformed polymer with C₁-C₂₀ alkanol. Average diameters of the dispersed polymer may range from about 0.001 micron to about 120 micron, preferably from about 0.01 micron to about 1 micron, optimally from about 0.1 micron to about 0.5 micron.

Number average molecular weight for these polymers may range from about 1,000 to about 1,000,000, preferably from about 2,000 to about 500,000, optimally from about 5,000 to about 20,000.

A variety of techniques well-known in the art can be used to prepare latexes of water-insoluble polymer particles which are useful opacifiers. These include, inter alia, hatch, semi-continuous and seeded emulsion polymerization techniques.

Particularly preferred opacifiers useful in the present invention are latexes presently commercially available under the trademark ACUSOL (ex. Rohm & Haas Inc.). These latexes are characterized by pH of about 2 to about 3, having approximately 40% solids in water, with particle size of about 0.1 to about 0.5 micron. Specific ACUSOL. polymers include ACUSOL OP301 described as being a latex of a styrene/acrylate polymer, ACUSOL OP302 described as being a latex of a styrene/acrylate/divinylbenzene copolymer, ACUSOL OP303 described as being a latex of a styrene/acrylamide copolymer, ACUSOL OP305 described as being a latex of a styrene/PEG-10 maleate/nonoxynol-10 maleate/acrylate copolymer and a styrene/acrylate/PEG-10 dimaleate copolymer. Further preferred latexes useful in the present invention include those styrene/polyvinylpyrrolidone co-polymers and styrene/acrylic emulsions. Such include styrene/polyvinylpyrrolidone co-polymers which can be used include, for example. POLECTRON 430 (ex. ISP Technologies, Inc.), as well as sodium styrene/acrylate/divinyl-benzene co-polymer and ammonium nonoxynol-4 sulfate; sodium styrene/PEG-10 maleate/nonoxynol-10 maleate/acrylates co-polymer and ammonium nonoxynol-4 sulfate; styrene/acrylamide co-polymer and ammonium nonoxynol-4 sulfate; styrene/acrylates co-polymer and sodium lauryl sulfate and octoxynol-9; sodium styrene/acrylates co-polymer and sodium lauryl sulfate and tridecath-7 sodium methacrylate/styrene co-polymer and sodium lauryl sulfate and tridecath-7 and sodium lauryl diphenyloxide-disulfonate and sodium styrene/acrylates co-polymer (ex CSA, line, Greenville S.C.). A particularly preferred opacifier is OPULYN 303B (ex, Robin & Haas Inc.) described, to be styrene/acryl amide emulsion.

The opacifier constituent of the invention is suitably present in amounts of up to about 5% wt., preferably are present in amounts of from about 0.01-5% wt., preferably are present in amount from about 0.1% wt. to about 1.2% wt, and most preferably are present in amounts of from about 0.1% wt, to about 1% wt., based on the total weight of the topical germicidal composition of which it forms a part. Concurrently the amount of the of the water-insoluble polymer present in the opacifier constituent may range from about 0.01 to about 90%, preferably from about 0.1 to about 60%, optimally from about 10 to about 50% by weight of the opacifier constituent.

Water is also necessarily present in the topical germicidal compositions, and provides to 100% by weight of the compositions of the invention. The water may be tap water, but is preferably distilled and is most preferably deionized water or “soft.” water. If the water is tap water, it is preferably substantially free of any undesirable impurities such as organics or inorganics, especially minerals salts which are present in hard water which may thus undesirably interfere with the operation of the constituents present in the topical germicidal compositions according to the present invention. When present, water may be present in various amounts of up to about 30% wt, of the total weight of the composition of which it forms a part, although it is frequently present in reduced amounts, e.g., 29% wt., 28% wt., 27% wt., 26% wt., 25% wt. based on the product form and further based on the total weight of the composition of which it forms a part. Advantageously water is included in the compositions in amounts of at least 10% wt, and preferably (and in order of increasing preference) at least 12% wt., 13% wt., 14% wt. 15% wt., 16% wt., 17% wt., 18% wt., 9% wt., 20% wt., 21% wt., 22% wt., 23% wt., 24% wt. and 25% wt, based on the total weight of the compositions of which water forms a part. Compositions of the invention in which no water is added to the constituents “as supplied” from their respective suppliers are also contemplated, as frequently one or more constituents may be supplied with an aqueous or aqueous/organic liquid carrier, in which case the water supplied as part of the one or more water comprising constituents may be used to calculate the total amount of water present in the overall topical germicidal compositions.

The topical germicidal compositions are preferably flowable, and depending upon the product limn may be provided in variety of viscosity ranges suited for a particular product type. For example, the topical germicidal compositions may be provided as thin “cosmetic milk” product format, and may have a viscosity as little at about 500 cP typically to about 2500 cP, while in a “lotion” product format may have somewhat higher viscosities as well, typically in the range of from about 2000 cP to about 10,000 cP, preferably in the range of about 2000 to about 8000 cP, while in a more Viscous form at such as a gel or thickened lotion may have a viscosity of about 9,000 cP or more, such as between about 10,000 cP and about 20,000 cP. Still moo viscous forms of topical germicidal compositions may be formed and are contemplated to be within the scope of the present invention, e.g., in the range of 10-100,000 cP at 25° C. as measured using conventional quantitative methods e.g., as measured at 20° C. or 25° C. by a Brookfield Type LVT or Type RVT viscometer using a standard spindle, (e.g., a #3 spindle) or alternately using a “T-bar” operating under a “heliopath” rather than rotational mode of operation as would be practiced with a spindle. The aforesaid viscosities are ones which may be based on the “as mixed” topical germicidal compositions but preferably are evaluated after at least 1 week, preferably at least 2 weeks of storage of a sample of the topical germicidal composition maintained at a temperature of at least 30° C. preferably at least 40° C. Certain preferred viscosities and storage time and temperature conditions are disclosed with reference to one or more of the examples.

The compositions exhibit a pH in the range of from about 4 to about 8, preferably a pH in the range of from about 5 to about 7., and most preferably between about 5 and about 6. Particularly preferred pH ranges are disclosed with reference to one or more of the examples. When necessary a pH adjusting agent or constituent may be used.

The compositions of the invention may include one or more further optional constituents which may be used to improve one or more aesthetic and/or technical characteristics of the composition of which they form a part. Typically they are included in only small amounts, and usually the total amount of any such optional constituents does not exceed 25% wt. of the topical germicidal compositions of which they form a part. In certain preferred embodiments of the invention, one or more of the following recited optional constituents may be considered as essential constituents according to a particular preferred embodiment. Such optional constituents include additives and adjuvants which are conventional in the cosmetic, pharmaceutical or dermatological field, such as hydrophilic or lipophilic gelling agents, hydrophilic or lipophilic active agents, emulsifiers, particulates, fillers, emollients, skin conditioning agents, preservatives, antioxidants, solvents especially organic solvents, pH adjusting agents, pH buffers, chealating agents, fragrances, fragrances or other materials which provide an aromatherapy benefit, fillers, preservatives, dyestuffs or colorants, and light stabilizers including UV absorbers.

The topical germicidal compositions may optionally but preferably contain a non ionic surfactant. By way of non-limiting examples of such nonionic surfactants include ethoxylated fatty alcohols, polyethoxylated fatty alcohols, glycerol mono-fatty acid esters fatty acid esters of polyethylene glycol, polyethoxylated sorbitan fatty acid esters, alkylglycosides, and alkylpolyolosides, although it is expected that any other surfactants, such anionic, nonionic, cationic, zwitterionic or amphoteric surfactant compound may also function as a useful co-emulsifier constituent. Such surfactants may be useful as emulsifier constituents.

A preferred surfactant constituent is an ethylene oxide condensed with sorbitan fatty acid esters. Such materials are presently commercially available under the tradename TWEEN (ex. ICI) and/or CRILL (ex. Croda) which include polyoxyethylene sorbitan monolaurate, polyoxyethylene sorbitan monopalmitate, polyoxyethylene sorbitan monostearate, polyoxyethylene sorbitan tri stearate, polyoxyethylene sorbitan monooleate, polyoxyethylene sorbitan trioleates which are available in a variety of grades, and with differing amounts of polyoxylethylene groups per molecule. Particularly preferred surfactants are described with reference to one or more of the examples. Such surfactants may be present in any effective amount, and when included, advantageously are present in amounts of from about 0.01% wt. to about 5% wt., preferably from about 0.25% wt. to about 2% wt., based on the total weight of the topical germicidal compositions of which they form a part. In certain particularly preferred embodiments the compositions of the invention necessarily include a surfactant constituent, whilst in other preferred embodiments surfactants are excluded from the compositions.

In certain preferred embodiments the inventive compositions exclude anionic soaps, as such may interfere with cationic compounds which may be present.

Optionally a thickener constituent may be present in compositions of the invention. One such thickener constituent is/are one or more thickener constituents based on crosslinked polycarboxylate and/or polyacrylate polymer thickeners; including those typically exhibit a molecular weight from about 500,000 to about 4,000,000, and generally have degrees of crosslinking of from about 0.25% to about 15%. Such crosslinked polycarboxylate and/or polyacrylate polymers may include in their structure other monomers besides acrylic acid such as ethylene and propylene, which act as diluents, and maleic anhydride which acts as a source of additional carboxylic groups. Such thickener constituents based on crosslinked polycarboxylate and/or polyacrylate polymer thickeners are widely commercially available and include, e.g., polycarboxylate polymers and/or polyacrylate polymers sold under trade names Carbopol®. Acrysol® ICS-1 and Sokolan®.

A further thickener constituent is one or more clay thickeners. Exemplary clay thickeners comprise, for example, colloid-forming clays, for example, such as smectite and attapulgite types of clay thickeners. The clay materials can be described as expandable layered clays, i.e., aluminosilicates and magnesium silicates. The term “expandable” as used to describe the instant clays relates to the ability of the layered clay structure to be swollen, or expanded, on contact with water. The expandable clays used herein are those materials classified geologically as smectites (or montmorillonite) and attapulgites (or polygorskites).

A further thickener constituent is one or more thickeners based on naturally occurring polysaccharide polymers such as xanthan gum, guar gum, locust bean gum, tragacanth gum, or derivatives thereof.

Any of the thickeners, when present, may be present in any amount which is found effective in achieving a desired degree of thickening. When present, advantageously such one or more thickener constituents may be present in amounts of from about 0.001% wt. to about 10% wt., preferably from about 0.01% wt, to about 5% wt., based on the total weight of the topical germicidal composition of which it forms a part.

In certain embodiments of the invention one or more of the recited thickeners are expressly excluded from the topical germicidal compositions.

In further embodiments of the invention at least one or more of the recited thickeners are expressly present within from the topical germicidal compositions concurrently with a film forming constituent based on cellulose or one or more cellulose derivatives.

The topical compositions of the invention may optionally comprise one or more emollients which provide softness to the topical germicidal compositions, Non-limiting examples of useful emollients include those, for example, compounds based on Guerbet alcohols based on fatty alcohols containing 6 to 18 and preferably 8 to 10 carbon atoms and other additional esters, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C₁₈₋₃₈ alkyl-hydroxycarboxylic acids with linear or branched C₆₋₂₂ fatty alcohols, more especially dioctyl malate, esters of linear and/or branched fatty acids with polyhydric alcohols (for example propylene glycol, dimer diol or trimer triol), triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, dicaprylyl carbonate (commercially available as Cetiol® CC), Guerbet carbonates based on fatty alcohols containing 6 to 18 and preferably 8 to 10 carbon atoms, esters of benzoic acid with linear and/or branched C₆₋₂₂ alcohols (for example, a product commercially available as Finsolv° TN), linear or branched, symmetrical or nonsymmetrical dialkyl ethers containing 6 to 22 carbon atoms per alkyl group such as, for example dicaprylyl ether (commercially available as Cetiol® OE), ring opening products of epoxidized fatty acid esters with polyols and hydrocarbons or mixtures thereof (commercially available as Cetiol® DD), propylheptyl caprylate (commercially available as Cetiol® SenSoft) as well as the compounds disclosed in published US Patent application 2009/0182046 the contents of which are herein incorporated by reference.

The topical antimicrobial composition may include a Polyquaternium compound or material, which are typically cationic polymer. Such materials, are, per se, well known to the art of topical compositions. Various grades of such cationic polymers may be used, inter alia: Polyquaternium 1; Polyquaterinum 2; copolymers of hydroxyethylcellulose and diallyldimethyl ammonium chloride commercially available as Polyquaternium 4 homopolymers of diallyldimethylammonium chloride commercially available as Polyquaternium 5; dimethyldiallylammonium chloride homopolymer commercially available as Polyquaternium 6; copolymers of diallyldimethylammonium chloride with acrylamide commercially available as Polyquaternium 7; the polymeric quaternary ammonium salt of methyl and steardyl dimethylaminoethyl methacrylate quaternized with dimethyl sulfate commercially available as Polyquaternium 8; the polymeric quaternary ammonium salt of polydimethylaminoethyl methacrylate quaternized with methyl bromide commercially available as Polyquaternium 9; a polymeric quaternary ammonium salt formed from the reaction of hydroxyethyl cellulose with a trimethylammonium substituted epoxide commerically available as Polyquaternium 10; a polymeric quaternary ammonium polymer formed by the reaction of vinyl pyrrolidine and dimethyl aminoethylmethacrylate commercially available as Polyquaternium 11; a polymeric quaternary ammonium salt prepared by the reaction of ethyl methacrylate/abietyl methacrylate/diethylaminoethyl methacrylate copolymer with dimethyl sulfate commercially available as Polyquaternium 12; a polymeric ammonium salt prepared by the reaction of ethyl methacrylate/oleyl methacrylate/diethylaminoethyl methacrylate copolymer with dimethyl sulfate commercially available as Polyquaterinum 12; a polymeric quaternary ammonium salt prepared by the reaction of ethyl methacrylate/oleyl, methacryalte/diethylamino ethyl methacrylate copolymer with dimethyl sulfate commercially available as Polyquaternium 13; Polyquaternium 14; the copolymer methacrylamide and betamethacrylyloxyethyl trimethyl ammonium chloride commercially available as Polyquaternium 15; the polymeric quaternary ammonium salt formed from methylvinylimidazolium chloride and vinylpyrrolidone commercially available as Polyquaternium 16; polymeric quaternary salts prepared by the reaction of adipic acid and dimethylaminopropylamine reached with dichloroethyl ether commerically available as Polyquaternium 17; a polymeric quaternary salt prepared by the reaction of azelaic acid and dimethylaminopropylamine reacted with dichloroethyl ether commercially available as Polquaternium 18; a polymeric quaternary ammonium salt prepared by the reaction of polyvinyl alcohol with 2,3-epoxypropylamine commercially available as Polyquaternium 19; a polymeric quaternary ammonium salt prepared by the reaction of polyvinyl octadecyl ether with 2,3-epoxypropylamine commercially available as Polyquaternium 20; copolymers of acrylic acid and dimethyldiallylammonium chloride commercially available as Polyquaternium 22; polymeric quaternary ammonium salts of hydroxyethyl cellulose reacted with lauryl dimethyl ammonium-substituted epoxide commercially available as Polyquaternium 24; a block copolymer formed by the reaction of Polyquaternium 2 and Polyquaternium 17 commercially available as Polyquaternium 27; a polymeric quaternary ammonium salt consisting of vinylpyrrolidone and dimethylaminopropyl methacrylamide monomers commercially available as Polyquaternium 28; chitosans reacted with propylene oxide and quaternized with epichlorohydrin commercially available as Polyquaternium 29; Polyquaternium 30; a polymeric quaternary ammonium salt prepared by the reaction of DMAPA acrylates/acrylic acid/acrylonitrogens copolymer with diethyl sulfate commercially available as Polyquaternium 31; Polyquaternium 32; Polyquaternium 33; Polyquaternium 34; Polyquaternium 35; Polyquaternium 36; Polyquaternium 37; polymeric quaternary ammonium salts of the terpolymer of acrylic acid/diallyldimethylammonium chloride/acrylamide commercially available as Polyquaternium 39; Polyquaternium 42; a copolymer of acrylamide, acrylamidopropyltrimonium chloride, 2-amidopropylacrylamide sulfonate and DMAPA polymers commercially available as Polyquaternium 43; a polymeric quaternary ammonium salt consisting of vinylpyrrolidone and quaternized imidazoline monomers commercially available as Polyquaternium 44; Polyquaternium 45; a polymeric quaternary ammonium salt prepared by the reaction of vinylcaprolactam and vinylpyrrolidone with methylvinylimidazolium commercially available as Polyquaternium 46; a polymer quaternary ammonium chloride formed by the polymerization of acrylic acid with methacrylamidopropyl trimethylammonium chloride and methylacrylate commercially available as Polyquaternium 47; a copolymer of methacryloyl ethyl betaine, 2-hydroxyethyl methacrylate and metacyloyl ethyl trimethyl ammonium chloride commercially available as Polyquaternium 48: a copolymer of methacryloyl ethyl betaine, PEG-9 methacrylate and methacryloyl ethyl trimethyl ammonium chloride commercially available as Polyquaternium 49; Polyquaternium 50; Polyquaternium 51; Polyquaternium 52; a copolymer of acrylic acid, acrylamide and methacrylamidopropyltrimonium chloride commercially available as Polyquaternium 53; a polymeric quaternary ammonium salt prepared by the reaction of aspartic acid and C6-C18 alkylamine with dimethylaminopropylamine and sodium chloroacetate commerically available as Polyquaternium 54; a polymeric quaternary ammonium chloride formed by the reaction of vinylpyrrolidone, dimethylaminopropyl methacrylamide and methacryloylaminopropyl lauryldimonium chloride commercially available as Polyquaternium 55; and a polymeric quaternary ammonium salt consisting of isophorone diisocyanate, butylene glycol and dihydroxyethyldimonium methosulfate monomers commercially available as Polyquaternium 56. Each of the foregoing are described in the literature, particularly in the International Cosmetic Ingredient Dictionary and Handbook, Volume 2 (9^{th} Edition, 2002), at pages 1311-1319. Other polyquaternium compounds although not specifically elucidated here may also be utilized in the present inventive compositions.

When present in the topical antimicrobial compositions, one or more Polyquaternium-type compounds or materials are advantageously present in amounts of from about from 0.001-5% wt., preferably in amounts from 0.01-2% wt., but are most desirably present in reduced weight percentages from about 0.05-1% wt based on the total weight of the topical antimicrobial composition of which they form a part.

A particularly preferred material which includes each of a Polyquaternium compound, an emollient, and a surfactant, is a material which is presently commercially available as Cosmedia® Triple C (ex. Cognis) which is described as a blend of materials comprising 55-60% wt of ethanaminium, N,N,N-trimethyl-2-((2-methyl-1-oxo-2-propenyl)oxy), chloride, homopolymer, and 30-40% wt of dioctyl carbonate, and 1-10% wt. of a C₁₀-C₁₆ alkylpolyglycoside, and 0-10% wt. of water.

Surprisingly the inventors have found than when both a Polyquaternium-type compounds are present in the compositions concurrently with an anionic opacifier constituent, the combination of these two materials when present in controlled amounts does not undesirably form precipitates which may render the compositions unattractive or unsaleable from a consumer standpoint. Preferred such Polyquaternium-type compounds and anionic opacifier constituents, and their respective proportions, are disclosed with reference to one or more of the Examples.

The topical antimicrobial compositions may include a cosmetic particulate, which may be any particulate material which is a solid at room temperature (approx. 20° C.) temperature and atmospheric pressure, which does not deleteriously react chemically with balance of the constituents of the in inventive composition. Advantageously the cosmetic particulate is insoluble in balance of the constituents of the topical antimicrobial compositions, particularly when the compositions are brought to a temperature above room temperature and especially to a temperature of at least 50° C. and preferably at least 60° C. for at least 24 hours, preferably for at least 48 hours. Desirably the cosmetic particulate constituent exhibits a melting temperatures of at least 70° C., preferably at least 100° C., more preferably at least 120° C., and most preferably at least 130° C. The cosmetic particulate composition may be absorbent or non-absorbent with respect to one or more of the remaining constituents of the inventive compositions of which they form a part.

Advantageously the cosmetic particulate constituent may be mineral or organic, lamellar, spherical, viz., beads, or oblong. They may have a generally regular geometry, such as in the case of spheres or rods, or they may have an irregular geometry such as crushed particulate materials. Exemplary materials useful for the cosmetic particulate constituent include: inorganic particulate particles formed from talc, mica, silica, kaolin, boron nitride, carbonates such as precipitated calcium carbonate, magnesium carbonate and magnesium hydrocarbonate, hydroxyapatite, hollow silica microspheres, glass microcapsules, and ceramic microcapsules, inorganic pigments and mixtures thereof. Exemplary materials useful ho the cosmetic particulate constituent include: organic particulate particles formed from polyamide powders, such as polyamides (Nylons), polyethylenes, polypropylenes polyesters, acrylic polymers such a polymethyl methacrylate, polytetrafluoroethylene (Teflons.), as well as crystalline and microcrystalline waxes derived from plants, mineral oils or petroleum, hollow polymer microspheres such as those formed from polyvinylidene chloride/acrylonitile, starches, alginates, organic dyestuffs or pigments, and mixtures thereof. Mixtures of two or more cosmetic panicles may be used to provide the cosmetic particulate constituent. Preferred as the cosmetic particulate constituent are materials which provide an exfoliating benefit.

Preferably, these cosmetic particulates have an apparent diameter in the range of from about 100 to about 1000 μm, preferably from about 100 to about 600 μm and most preferably from about from about 250 to about 600 μm. An apparent diameter corresponds to the diameter of the circle in which the elementary particle is inscribed along its smallest dimension (thickness for lamellae).

A preferred class of cosmetic particulate materials are based on synthetically occurring or synthetic waxes inclusive of microcrystalline waxes. Exemplary useful waxes include any of those which are generally useful used in cosmetics and dermatology. Exemplary waxes of natural origin, include for instance beeswax, carnauba wax, candelilla wax, ouricoury wax, Japan wax, cork fibre wax or sugar cane wax, paraffin wax, lignite wax, microcrystal line waxes, lanolin wax, montan wax, ozokerites, hydrogenated oils, liar instance hydrogenated jojoba oil. Exemplary waxes of synthetic origin include for instance polyethylene waxes derived from the polymerization of ethylene, waxes obtained by Fischer-Tropsch synthesis, esters of fatty acids and of glycerides that are solid at 50° C. preferably at 60° C. or higher temperatures, and silicone waxes, for instance alkyl, alkoxy, and/or esters of poly(di)methylsiloxane that are solid at 50° C. preferably at 60° C. or higher temperatures. These waxes may be formed particulates, e.g., beads or spheres according to conventional methods.

The cosmetic particulate constituent of the invention may be provided in any effective amount, but desirably is present in amount which are aethetically pleasing to the user of the composition. The cosmetic particulate constituent is made of individual cosmetic particulate materials which may be of a uniform chemical or physical composition, and/or of a uniform size or dimension and/or of a uniform color but this is not a necessity and mixtures or different individual cosmetic particulate materials which may be differentiated on the basis of chemical and/or physical composition, and/or size or dimension and/or color may be provided as the cosmetic particulate constituent of the invention. If included in the compositions of the invention, the cosmetic particulate constituent of the invention may be provided in any effective amount, advantageously from at least 0.01% wt., preferably at least 0.05% wt, and most preferably at least 0.1% wt of the topical antimicrobial composition. Similarly advantageously the cosmetic particulate constituent is present in not more than 10% wt., preferably not more than 5% wt, and yet more preferably not more than 2% wt., and most preferably not more than 2% wt of the topical antimicrobial composition of which it forms a part.

The topical antimicrobial compositions may include one or more fillers in the Rum of powders. By way of non-limiting examples these powders include chalk, talc, kaolin, starch, smectite clays, chemically modified magnesium aluminum silicate, organically modified montmorillonite day, hydrated aluminum silicate, fumed silica, aluminum starch octenyl succinate and mixtures thereof. When present in a composition, the one or more fillers may be present in amounts of up to about 0.5% wt., preferably are present in amounts of from about 0.001% wt. to about 5% wt based on the total weight of the topical composition of which it forms a part.

The compositions of the invention may optionally include one or more polysiloxanes which are commonly used and often interchangeably referred to as silicone emulsifiers. Such silicone emulsifiers include polydiorganosiloxlinepolyoxyalkylene copolymers containing at least one polydiorganosiloxane segment and at least one polyoxyalkylene segment. The polyoxyalkylene segments may be bonded to the polydiorganosiloxane segments with silicon-oxygen-carbon bonds and/or with silicon-carbon bonds. The polydiorganosiloxane segments of consist essentially of siloxane units which are interlinked by Si—O—Si linkages and which have the formula:

R_{b}SiO_{(4{b})/2}

The value of b may range from 0 to 3 for said siloxane units with the provision that there is an average of approximately 2, i.e. from 1.9 to 2.1 R radicals for every silicon in the copolymer. Suitable siloxane units thus include R₃SiO_{1/2}, R₂SiO_{2/2}, RSiO_{3/2}, and SiO_{4/2} siloxane units taken in such molar amounts so that b has an average value of approximately 2 in the copolymer. Said siloxane units may be arranged in linear, cyclic and/or branched fashion. The R radicals may be any radical selected from the group consisting of methyl, ethyl, vinyl, phenyl, and a divalent radical bonding a polyoxyalkylene segment to the polydiorganosiloxane segment. At least 95 percent of all R radicals are methyl radicals: preferably there is at least one methyl radical bonded to each silicon atom in (d). Divalent R radicals preferably contain no more than 6 carbon atoms. Examples of divalent R radicals include —O—, —CₘH₂ₘO—, —CₘH₂ₘ— and —CₘH₂ₘCO₂— where as is an integer greater than zero, illustrative of the siloxane units that make up the polydiorganosiloxane segments are the following, where Me denotes methyl and Q denotes said divalent R radical and bonded polyoxyalkylene segment: R₃SiO_{1/2} units such as Me₃SiO_{1/2}, Me₂(CH₂═CH)SiO_{1/2}, Me₂(C₆H₅)SiO_{1/2}, Me(C₆H₅)(CH₂═CH)SiO_{1/2}, Me(CH₃CH₂)SiO_{1/2}, Me₂QSiO_{1/2}, MeQ₂ SiO_{1/2}, Q₃SiO_{1/2}, Q₂(CH₃CH₂)SiO_{1/2}, and Me(C₆H₅)(Q)SiO_{1/2}; R₂SiO_{2/2} units such as Me₂SiO_{2/2}, Me(C₆H₅)SiO_{2/2}, Me(CH═CH)SiO_{2/2}, (C₆H₅)₂SiO_{2/2}, MeQSiO_{2/2}, and Q(C₆H₃)SiO_{2/2}; RSiO_{3/2} units such as MeSiO_{3/2}, C₆H₅SiO_{3/2}, CH₂═CHSiO_{3/2} CH₃CH₂SiO_{3/2} and QSiO_{3/2}; and SiO_{4/2} units.

Volatile linear silicones including polydimethylsiloxane and dimethicones may also be present as silicone emulsifiers in compositions according to the invention.

Also useful as silicone emulsifiers in the inventive compositions are one or more compounds which may be represented by the structure:

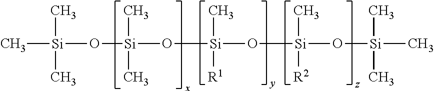

wherein

R¹ represents a C₁-C₃₀ straight chained, branched or cyclic alkyl group,

R² represents a moiety selected from:

—(CH₂)ₙ—O—(CH₂CHR³O)ₘ—H

and

(CH₂)ₙ—O—(CH₂CHR³O)ₘ—(CH₂CHR⁴O)ₚ—H

in which n represents an integer from about 3 to about 10, R3 and R4 are sleeted from hydrogen and C1-C6 straight chain, or branched chain alkyl groups with the proviso that R³ and R⁴ are not simultaneously the same, each of in, p, x and y are independently selected from integers of zero or greater, such that the molecule has a molecular weight of between about 200 to about 20,000,000 and wherein both in and p are not both simultaneously zero, and z is selected from integers of or greater.

If included in the compositions of the invention, the silicone emulsifiers may be provided in any effective amount, advantageously from at least 0.01% wt., preferably at least 0.05% wt, of the composition. Advantageously the silicone emulsifiers, when Present, are present in amounts of not more than 5% wt, and yet more preferably not more than 2% wt, and most preferably not more than 2% wt of the composition of which it forms a part.

The topical germicidal compositions may include one or more powders or pulvurent materials. These powders include mica, chalk, talc. Fullers earth, kaolin, starch, silica, silicates, hydrated aluminum silicate, fumed silica, aluminum starch octenyl succinate as well as comminuted or particulate polymers such as particles of polyamides (Nylons), polyalkyleneterephtalates (PET, PBT), polyolelins (PE) or fluoropolymers (polytetrafluomethylene) as well as mixtures of two or more thereof. The inclusion of one or more powders in the in compositions may provide in improved tactile benefit and/or may act to absorb as part of one or more of the hydrophobic constituents present in the composition and/or may provide an opacifying effect to the compositions. Preferred powders are those based on inorganic materials, e.g., silica, silicates and talc. Such are typically provided to the topical germicidal compositions as finely divided panicles. While such powders may be included in any effective amount, when present they are advantageously included in amounts of between about 0.01% wt. to about 5% wt., preferably between about 0.25% wt. to about 2% wt., based on the total weight of the topical germicidal composition of which they form a part.

The topical germicidal compositions may include one or more high molecular weight polyethylene glycol polymers (also referred to as poly(ethylene oxide) or polyoxyethylene), (“PEG”) having a molecular weight of at least about 100 preferably at least about 200, yet more preferably at least about 300, with yet higher molecular weights of about 1000 and even more also contemplated as being useful. Such are typically provided in a solid, or pulvurent form and depending upon the molecular weight may be kit east partially soluble in the inventive compositions. Such materials are widely commercially available under various tradenames, inter alia, Polyox® materials (ex, Dow Chem. Co.). While such high molecular weight polyethylene glycol polymers may be included in any effective amount, when present they are advantageously included in amounts of between about 0.01% wt. to about 5% wt., preferably between about 0.25% wt. to about 4% wt., based on the total weight of the topical germicidal composition of which they form a part.

The topical germicidal compositions may include which comprise one or more paraffinic hydrocarbons and/or preparations containing the same. Such paraffinic hydrocarbons may include one or both of linear and/or branched paraffinic hydrocarbons. Mixtures of branched hydrocarbons especially as isoparaffins form a further particularly preferred form of a useful hydrocarbon solvent of the invention. Particularly useful technical grade mixtures of isoparaffins include mixtures of isoparaffinic organic solvents having a relatively narrow boiling range. Examples of these commercially available isoparaffinic car solvents include those consisting substantially of linear isoparaffins, e.g., those commercially available as Norpar® solvents (ex. ExxonMobil Corp.) as well as those containing branched isoparaffins, e.g., those commercially available as Isopar® solvents (ex. ExxonMobil Corp.) Examples of the latter include Isopar® C described to be primarily a mixture of C₇-C₈ isoparaffins, Isopar® J described to be primarily a mixture of C₁₁-C₁₂ isoparaffins, Isopar® M described to be primarily a mixture of C₁₃-C₁₄ isoparaffins, and Isopar® V described to be primarily a mixture of C₁₂-C₂₀ isoparaffins. Further, other preparations which include a significant proportions of one or more isoparaffins may also be utilized. Such include, for example, SiClone® SR-5, (ex. Presperse LLC, Somerset, N.J. (USA)) which is described to be a technical mixture of C₁₃-C₁₆ isoparaffins, C₁₂-C₁₄ isoparaffins, with a C₁₃-C₁₅ alkane constituent, which technical mixture is marketed as a substitute for cyclomethicone in cosmetic formulations, yet is 100% silicone-free.

While such paraffinic hydrocarbons and/or preparations containing the same may be included in any effective amount, when present they are advantageously included in amounts of between about 0.01% wt. to about 5% wt., preferably between about 0.1% wt. to about 2% wt., based on the total weight of the topical germicidal composition of which they form a part.

The topical germicidal compositions may include one or more preservatives. Exemplary useful preservatives include compositions which comprise parabens, including methyl parabens and ethyl parabens, glutaraldehyde, formaldehyde, 2-bromo-2-nitropropoane-1,3-diol, 5-chloro-2-methyl-4-isothiazolin-3-one 2-methyl-4-isothiazoline-3-one, and mixtures thereof. Further suitable preservatives include those marketed as: KATHON CG/ICP, KATHON CG/ICP II (ex, Rohm and Haas Inc.), PROXEL (ex. Zeneca). SUTTOCIDE A (ex, Sutton Laboratories) and TEXTAMER 38AD (ex. Calgon Corp.) When present the preservative is included in any amount found to be effective in retarding or inhibiting the grown of undesired microorganisms in the topical germicidal compositions, particularly during storage for several months at room temperature. The preservative composition is advantageously present in amounts of up to about 1.5% wt., preferably from about 0.00001% wt. to about 0.5% wt., most from about 0.0001% wt. to 0.25 wt. based on the total weight of the topical composition of which it forms a part. Usually however, in light of the high alcohol content such preservatives are not required and are advantageously omitted.

The topical germicidal compositions may include a fragrance constituent, which may be based on natural and synthetic fragrances and most commonly are mixtures or blends of a plurality of such fragrances, optionally in conjunction with a carrier such as an organic solvent or a mixture of organic solvents in which the fragrances are dissolved, suspended or dispersed. When present in a composition, the fragrance constituent may be present in any effective amount such that it can be discerned by a consumer of the topical germicidal composition, however is advantageously present in amounts of up to about 5% wt., preferably from about 0.00001% wt. to about 1.5% wt., most preferably from about 0.0001% wt. to 0.25% wt. based on the total weight of the topical composition of which it forms a part.

The inventive topical germicidal compositions may include one or more colorants, e.g, dyes or pigments which are known to the art be useful in cosmetic, or topical compositions which may be used to impart a desired color or tint to the inventive compositions. Exemplary colorants include pigments, inter ilia, inorganic red pigments, such as iron oxide., iron hydroxide and iron titanate; inorganic brown pigments, such as gamma-iron oxide; inorganic yellow pigments, such as iron oxide yellow and loess; inorganic black pigments, such as iron oxide black and carbon black; inorganic violet pigments, such as manganese violet and cobalt violet; inorganic green pigments, such as chromium hydroxide, chromium oxide, cobalt oxide and cobalt titanate; inorganic blue pigments, such as Prussian blue and ultramarine blue; lakes of tar pigments; lakes of natural dyes; and synthetic resin powder complexes of the inorganic pigments as recited above. Advantageously one or more colorants may be added in amounts of about 0.001% wt, to about 0.1% by weight, based on the total weight of the composition of which the colorant(s) forms apart.

The topical germicidal compositions of the invention may one or more essential oils which are selected to provide a so-called “aromatherapy benefit” or “holistic benefit” to the user. Essential oils are complex mixtures of different organic molecules, such as terpenes, alcohols, esters, aldehydes, ketones and phenols. Such essential oils are frequently extracted from naturally occurring botanical sources such as flowers, stems, leaves, roots and barks of aromatic plants. While essential oils may be used singly, it is also common to utilize blends of essential oils in order to provide a conjunctive aroma benefit, aromatherapy benefit, holistic benefit and possibly a therapeutic benefit as well.

Preferred essential oils providing an aromatherapy benefit for use in the topical germicidal compositions of the present invention include one or more selected from chamomile oil, lavendin oil, lavender oil, grapefruit oil, lemon oil, line oil, mandarin orange oil, orange flower oil and orange oil. Chamomile oil may be used to promote both a fresh, clean and attractive scent and possibly provide a stress-relaxing benefit to the user of the topical composition. Lavender oil, and lavendin, may be used to promote both a fresh and attractive scent and possibly also provide, a stress-relaxing benefit to the user of the topical composition. One or more of grapefruit oil, lemon oil, line oil, mandarin orange oil, orange flower oil and orange oil provide a dean citrus scent and may possibly impart a perceived therapeutic benefit as well when used.

As used in the present invention, these one or more essential oils providing an aromatherapy benefit or holistic benefit are present in an amount about 0.00001 wt. % to about 1 wt. %, preferably from about 0.00005 wt. % to about 0.75 wt. %, and more preferably from about 0.0001 wt, % to about 0.5 wt % of the total weight of the composition. It is to be understood that these one or more essential oils providing an aromatherapy benefit may be used with our without the optional fragrancing constituent recited previously and may be used wholly or partially in place of said fragrancing constituent.

The topical germicidal compositions may include one or more antioxidant constituents; certain of these antioxidant constituents may additionally provide an anti-wrinkling benefit to the skin or other topical treatment benefit. Examples of antioxidants include but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, glutathione, and ascorbic acid and ascorbic acid derivatives (e.g., ascorbyl palmitate and ascorbyl polypeptide), as well as oil-soluble antioxidants such as butylated hydroxytoluene, retinoids, tocopherols e.g., tocopherol acetate, tocotrienols, and ubiquinone, natural extracts containing antioxidants such as extracts containing flavonoids and isoflavonoids and their derivatives, extracts containing resveratrol and the like, as well as certain natural extracts e.g., grape seed, green tea, pine bark, propolis, and the like. When present the total amount of such antioxidants are usually not in excess of 5% wt, preferably from 0.0001-4% wt. based on the total weight of the topical germicidal compositions of which it forms a pan. In certain preferred embodiments one or more antioxidants constituents are necessarily present.

Optionally the topical germicidal compositions may include one or more vitamins. Examples of vitamins which can be added include vitamin A, such as vitamin A oil, retinol, retinyl acetate and retinyl palmitate; vitamin B, including vitamin B₂ such as riboflavin, riboflavin butyrate and flavin adenine nucleotide, vitamin B₆ such as pyridoxine hydrochloride, pyridoxine dioctanoate and pyridoxine tripalmitate, vitamin B₁₂ and its derivatives, and vitamin B₁₅ and its derivatives; vitamin C, such as L-ascorbic acid, L-ascorbic acid dipalmitic ester, sodium (L-ascorbic acid)-2-sulfate and dipotassium L-ascorbic acid diphosphate vitamin D, such as ergocalciferol and cholecarciferol; vitamin E, such as alpha-tocopherol, beta-tocopherol, gamma-tocopherol, tocopheryl acetate, dl-alpha-tocopheryl nicotinate and dl-alpha-tocopheryl succinate. When present, in accordance with certain of the preferred embodiments, one or more vitamins may be included in effective amounts, advantageously from 0.0001-1% wt., preferably from 0.001-0.75% wt. based on the total weight of the topical germicidal compositions of which it forms a part.

The topical germicidal compositions may include one or more light stabilizers as well as UV absorbers or sunscreen constituents. Such materials are known to be useful in cosmetic or topical compositions and impart a degree of stability to the compositions which may comprise one or more components which may be deleteriously affected when exposed to certain sources of light. e.g., sunlight, fluorescent light sources. Other such materials are known to stabilize or improve the effect of colorants which may be present in the compositions. Any cosmetically acceptable material or compound which provides protection for one or more of the constituents in the inventive compositions from photolytic degradation or photo-oxidative degradation may be used. Examples include: triazines including s-triazine, triazine derivatives 2,4,6-trianilino-(p-carbo-2′-ethyl-1′-hexyloxy)-1,3,5-triazine, anisoniazine, ethylhexyltriazone, diethylhexylbutamidotriazone; benzotriazoles and derivatives; esters of benzalmalonic acid; sulphonic acid derivatives of 3-benzylidencamphen; cinnamic acid and cinnamic acid amides, esters of cinnamonic acid; propane-1,3-diones; phenylbenzimidazoles and sulfonated benzimidazoles; salicylic acid derivatives including esters of salicylic acid, e.g., ethylhexyl salicylate, dipropylene glycol salicylate, TEA salicylate, salicylic acid 2 ethylhexylester, salicylic acid 4-isopropyl benzylester, salicylic acid homomenthylester: compounds or derivatives of compounds based on benzylidenecamphor, and the like. Any of the foregoing materials provided as acids may used in free acid form or as a salt thereof, e.g., an alkali, alkaline earth, ammonium, alkyl ammonium, alkanolammonium salt form thereof. When present, the one or more light stabilizers as well as UV absorbers may be included in any effective amount; advantageously such materials are present in amounts of from 0.0001-1% wt., preferably from 0.001-0.5% wt. based on the total weight of the topical germicidal composition of which it forms a part.

The inventive topical germicidal compositions may comprise further one or more further antimicrobial agents. Such further antimicrobial agent is/are one or more compounds which provide an appreciable germicidal benefit. Such further antimicrobial agent desirably provides an effective antimicrobial benefit to treated dermal surfaces, e.g., hands, arms, etc.

The further antimicrobial agent may be include one or more cationic surfactant constituents, especially preferably one or more cationic surfactants which provide an appreciable germicidal benefit. Non-limiting examples of preferred cationic surfactant compositions which may be included in the treatment compositions are those which provide an appreciable germicidal benefit, and especially preferred am quaternary: ammonium compounds and salts thereof, which may be characterized by the general structural formula:

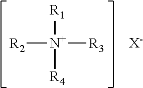

where at least one of R₁, R₂, R₃ and R₄ is a alkyl, aryl or alkylaryl substituent of from 6 to 26 carbon top the entire caption portion of the molecule has a molecular weight of at least 165. The alkyl substituents may be long-chain alkyl, long-chain alkoxyaryl, long-chain alkylaryl, halogen-substituted long-chain alkylaryl, long-chain alkylphenoxyalkyl, arylalkyl, etc. The remaining substituents on the nitrogen atoms other than the abovementioned alkyl substituents are hydrocarbons usually containing, no more than 12 carbon atoms. The substituents R₁, R₂, R₃ and R₄ may be straight-chained or may be branched, but are preferably straight-chained, and may include one or more amide, ether or ester linkages. The counterion X may be any salt-forming anion which permits water solubility or water miscibility of the quaternary ammonium complex. Preferred quaternary ammonium compounds which act as germicides according to the foregoing formula are those in which R₂ and R₃ are the same or different C₈-C₁₂alkyl, or R₂ is C₁₂₋₁₆alkyl, C₈₋₁₈alkylethoxy, C₈₋₁₈alkylphenolethoxy and R₃ is benzyl, and X is a halide, for example chloride, bromide or iodide, or is a methosulfate anion. The alkyl groups recited in R₂ and R₃ may be straight-chained or branched, but are preferably substantially linear.

The further antimicrobial agent may include one or more of: pyrithiones such as zinc pyrithione, halohydantoins such as dimethyldimethylol hydantoin, methylchlorisothiazolinone/methylisothiazolinone sodium sulfite, sodium bisulfite, imidazolidinyl urea, diazolidinyl urea, benzyl alcohol, 2-bromo-2-nitropropane-1,3-diol, formalin (formaldehyde), iodopropenyl butylcarbamate chloroacetamide, methanamine, methyldibromonitrile glutaronitrile, glutaraldehyde, 5-bromo-5-nitro-1,3 dioxane, phenethyl alcohol, o-phenylphenol/sodium o-phenylphenol, sodium hydroxymethylglycinate, polymethoxy bicyclic oxazolidine, dimethoxane, thimersal dichlorobenzyl alcohol, captan, chlorphenenesin, dichlorophene, chlorbutanol, glyceryl laurate, halogenated diphenyl ethers such as 2,4,4-trichloro-2-hydroxy-diphenyl ether (Triclosan®) and 2,2-dihydroxy-5,5-dibromo-diphenyl ether, phenolic antimicrobial compounds such as mono- and poly-alkyl and aromatic halophenols, such as p-chlorophenol, methyl p-chlorophenol, 4-chloro-3,5-dimethyl phenol. 2,4-dichloro-3,5-dimethylphenol, 3,4,5,6-terabromo-2-methylphenol, 5-methyl-2-pentylphenol, 4-isopropyl-3-methylphenol, para-chloro-meta-xylenol, dichloro meta xylenol, chlorothymol, and 5-chloro-2-hydroxydiphenylmethane, resorcinol and its derivatives, bisphenolic compounds such as 2,2-methylene his (4-chlorophenol) and his (2-hydroxy-5-chlorobenzyl)sulphide, benzoic esters (parabens), halogenated carbanilides such as 3-trifluoromethyl-4,4′-dichlorocarbanilide (Triclocarban), 3-trifluoromethyl-4,4-dichlorocarbanilide and 3,3,4-trichlorocarbanilide. The further antimicrobial agent may include one or more of: biguanides such as polyhexamethylene biguanide, p-chlorophenyl biguanide; 4-chlorobenzhydryl biguanide, 1,6-bis-(4-chlorobenzylbiguanido)-hexane (Fluorhexidine®), halogenated hexidine including, but not limited to, chlorhexidine (1,1′-hexamethylene-bis-5-(4-chlorophenyl biguanide) (Chlorohexidine®), as well as salts of any of the foregoing, e.g. polyhexamethylene biguanide hydrochloride.

Desirably, when present, such further antimicrobial agent may be included in the inventive compositions in any effective amount. Advantageously such amounts are from about 0.0001-2% wt., but preferably are from about 0.01-1% wt. of the topical germicidal composition of which they form a part.

In certain particularly preferred embodiments, the inventive compositions expressly exclude such a further antimicrobial constituent.

In order to adjust the pH of the inventive compositions, one or more pH adjusting agents as well as one or more pH buffers may optionally be included in the topical antimicrobial compositions in effective amounts. By way of non-limiting example pH adjusting agents include phosphorus containing compounds, monovalent and polyvalent salts such as of silicates, carbonates, and borates, certain acids and bases, tartrates and certain acetates. Further exemplary pH adjusting agents include mineral acids, basic compositions, and organic acids, which are typically required in only minor amounts. By way of further non-limiting example pH buffering compositions include the alkali metal phosphates, polyphosphates, pyrophosphates, triphosphates, tetraphosphates, silicates, metasilicates, polysilicates, carbonates, hydroxides, and mixtures of the same. Certain salts, such as the alkaline earth phosphates, carbonates, hydroxides, can also function as buffers. It may also be suitable to use as buffers such materials as aluminosilicates (zeolites), borates, illuminates and certain organic materials such as gluconates, succinates, maleates, and their alkali metal salts. When present, the pH adjusting agent, especially the pH buffers are present in an amount effective in order to maintain the pH of the inventive composition within a desired or a target pH range. Advantageously they may be included in generally minor amounts such as from 0.001-1.5% wt. but desirably are present in amounts from 0.01-1% wt. Exemplary and preferred pH buffers and pH adjusting agents are described with reference to one or more of the following Examples.

The inventive topical antimicrobial compositions may include one or more chelating agents. Exemplary useful chelating agents include those known to the art, including by way of non-limiting example; aminopolycarboxylic acids and salts thereof wherein the amino nitrogen has attached thereto two or more substitutent groups. Preferred chelating agents include acids and salts, especially the sodium and potassium salts of ethylenediaminetetraacetic acid, diethylenetriaminepentaacetic acid, N-hydroxyethylethylenediaminetriacetic acid, and of which the sodium salts of ethylenediaminetetraacetic acid may be particularly advantageously used. Such chelating agents may be omitted, or they may be included in generally minor amounts such as from 0.001-0.5% wt, based on the weight of the chelating agents and/or salt forms thereof. Desirably, such chelating agents are included in the present inventive composition in amounts from 0.01-0.5% wt., but are most desirably present in reduced weight percentages from about 0.01-0.2% wt.

In a further aspect, the present invention also contemplates a method for providing a cleaning and/or providing an germicidal benefit to skin or other topical surface which method contemplates the topical application of the aqueous topical germicidal compositions as described herein in a cleaning and/or germicidally effective amount. Preferably according to the foregoing method, a germicidal benefit is provided to the skin or other topical surface to which the composition has been applied. Preferred embodiments of the topical germicidal compositions exhibit good germicidal efficacy of undesired microorganisms, e.g., S. aureus, E. coli P. auruginosa, as well as E. hirae on dermal (viz., skin, body) surfaces. Advantageously the topical germicidal compositions exhibit antimicrobial efficacy against one or more of certain gram positive pathogens, certain gram negative pathogens, certain viruses, certain fungi and/or certain mold.

While the topical germicidal compositions disclosed herein find a primary use in application to the skin to provide a cleaning and/or germicidal benefit thereto and is contemplated as being provided in a dispenser for use in such a treatment, it is to be understood that this is not to be understood as a limiting definition and that other brims and other uses of the present inventive composition, such as face lotion, milky lotion, cream, face cleansing cream, massage materials, liquid toilet soap, as well as in hair care products such as shampoo, rinse or other hair or scalp treatment are expressly contemplated as being within the scope of the present invention. The topical germicidal compositions of the invention are beneficially formulated as a pourable lotion, a cosmetic milk, a liquid or a spray, but may also be formulated as a more viscous a cream or a gel, which may be transparent, translucent or opaque. In certain preferred embodiments the topical germicidal compositions is provided as a translucent or opaque composition.

The composition can be packaged in a suitable container to suit its viscosity and intended use by the consumer. For example, a lotion or cream can be packaged in a bottle, or can be packaged with a propellant in a propellant-driven aerosol device or alternately may be packaged in a container fitted with a manually operable pump. When the compositions of the invention have higher viscosities and is in the form of a paste, gel or cream it may conveniently be provided in a resealable container with a relatively wide opening, e.g., a jar, tin, tub or bottle with a removable and replaceable cap or cover. Forms of the composition which have low viscosities may be provided in bottles or flasks from which they be dispensed by pouring, or by pumping such as via a manually pumpable trigger pump or manually operable trigger spray pump. The inventive composition can be provided and stored in a non-deformable bottle but more preferably is provided in a squeezable container, such as a tube or deformable bottle which provides for easy dispensing of the composition by the consumer. Thus a further aspect of the invention provides a closed container containing the inventive composition as described herein.

It is to be further expressly understood that topical application of the topical germicidal compositions disclosed herein may be applied to the skin on any part of the body, including the skin on the face, neck, chest, back, aims, axilla, hands, legs, and scalp. The topical germicidal compositions disclosed herein may also be used on the hair. Preferably the topical germicidal compositions are not ingested or used on mucous tissues.

It is contemplated that in use, the consumer dispenses a quantity of the topical germicidal composition described herein and applied it to the skin or any other part of the body where they may be retained upon but are beneficially rubbed into the applied skin or other part of the body by the consumer to provide both a skin moisturization benefit concurrently with a germicidal benefit to the treated skin or other part of the body, Advantageously the thus applied topical germicidal composition is allowed to remain on the skin or other part of the body to which it has been applied, without any subsequent washing or rinsing. However, if desired by a consumer, the topical germicidal treatment compositions may be rinsed by the consumer under a stream of running water, e.g, in a shower or by immersion into water, e.g, a bath. Thus, a further aspect of the invention is directed to the use of the topical germicidal compositions as described herein.

The following examples below illustrate exemplary formulations as well as preferred embodiments of the invention. It is to be understood that these examples are provided by way of illustration only and that further useful formulations falling within the scope of the present invention and the claims may be readily produced by one skilled in the art without deviating from the scope and spirit of the invention.

### EXAMPLES

A number of topical germicidal compositions were produced according to the process described below, are described on Table 1 below. In the following compositions, the constituents were used “as supplied” from their respective suppliers and may constitute less than 100% wt. “actives”, or may have been supplied as constituting. 100% wt. “active” of the named compound, as indicated in the following Tables 1 and 2. The amounts indicated on Table 1 refer to % wt. of the “as supplied” named constituent used in a composition. Compositions which are comparative examples are identified by digits preceded with the letter “C”, while compositions according to the invention are identified by digits preceded with the letter “E”.

TABLE 1

| | C1 | E1 | E2 | E3 | E4 | E5 | E6 |
|---|---|---|---|---|---|---|---|
| ethanol (95%) | 66.0 | 66.0 | 66.0 | 66.0 | 66.0 | 68.0 | 66.0 |
| Methocel ® E4M | 0 | 0.1 | 0.25 | 0.5 | 1 | 0.25 | 0.1 |
| glycerine | 5.0 | 5.0 | 5.0 | 5.0 | 5.0 | 5.0 | 2.5 |
| Opulyn ® 303B (40%) | 0.5 | 0.5 | 0.5 | 0.5 | 0.5 | 0.5 | 0.5 |
| Cosmedia ® Triple C (90-100%) | 2.0 | 2.0 | 2.0 | 2.0 | 2.0 | 2.0 | 2.0 |
| Cetiol@ Sensoft | 2.0 | 2.0 | 2.0 | 2.0 | 2.0 | 2.0 | 2.0 |
| fragrance | 0.03 | 0.03 | 0.03 | 0.03 | 0.03 | 0.03 | 0.03 |
| aloe vera | 0.1 | 0.1 | 0.1 | 0.1 | 0.1 | 0.1 | 0.1 |
| chamomile extract | 0.1 | 0.1 | 0.1 | 0.1 | 0.1 | 0.1 | 0.1 |
| sodium hydroxide solution (10%) | 0.5 | 0.5 | 0.5 | 0.5 | 0.5 | 0.5 | 0.1 |
| d.i. water | 23.82 | 23.72 | 23.52 | 23.27 | 22.77 | 13.52 | 26.07 |
| pH | 5-6 | 5-6 | 5.83 | 5-6 | 5-8 | 5-6 | 5.72 |
| viscosity (cP) | 6000 | 18000 | 28000 | 40000 | 90000 | — | 18000 |

| | E7 | E8 | E9 |
|---|---|---|---|
| ethanol (95%) | 66 | 66 | 66 |
| Methocel ® E4M | 0.1 | 0.1 | 0.25 |
| glycerine | 2.5 | 2.5 | 5.0 |
| Opulyn ® 303B (40%) | 0.5 | 0.5 | 0.5 |
| Cosmedia ® Triple C (90-100%) | 2.0 | 2.0 | 2.0 |
| Cetiol ® Sensoft | 2.0 | 2.0 | 2.0 |
| SiClone ® SR-5 | 0.5 | 1.0 | 1.0 |
| fragrance | 0.03 | 0.3 | 0.3 |
| aloe vera | 0.1 | 0.1 | 0.1 |
| chamomile extract | 0.1 | 0.1 | 0.1 |
| sodium hydroxide solution (10%) | 0.1 | 0.1 | 0.1 |
| d.i. water | 26.07 | 25.57 | 22.92 |
| pH | 5.04 | — | 5.5 |
| viscosity (cP) | 22000 | — | 24000 |

| | E10 | E11 |
|---|---|---|
| ethanol (95%) | 66 | 66 |
| Methocel ® E4M | 0.1 | 0.1 |
| glycerine | 2.5 | 3.5 |
| Opulyn ® 303B (40%) | 0.5 | 0.5 |
| Cosmedia ® Triple C (90-100%) | 2.0 | 2.0 |
| Cetiol ® Sensoft | 2.0 | 2.0 |
| Polyox WSR 205 | 0.1 | 0.1 |
| fragrance | 0.03 | 0.03 |
| aloe vera | 0.1 | 0.1 |
| chamomile extract | 0.1 | 0.1 |
| sodium hydroxide solution (10%) | 0.1 | 0.1 |
| d.i. water | 26.47 | 25.47 |
| pH | 5.51 | 5.72 |
| viscosity (cP) | 12000 | 18000 |

| | E12 | E13 | E14 |
|---|---|---|---|
| ethanol (95%) | 66 | 66 | 66 |
| Methocel ® E4M | 0.1 | 0.1 | 0.25 |
| glycerine | 2.5 | 2.5 | 5 |
| Opulyn ® 303B (40%) | 0.5 | 0.5 | 0.5 |
| Cosmedia ® Triple C (90-100%) | 2.0 | 2.0 | 2.0 |
| Cetiol ® Sensoft | 2.0 | 1.0 | 2.0 |
| DC 200 fluid | 0.5 | 1.0 | 1.0 |
| SilSoft E-Pearl | — | — | — |
| fragrance | 0.03 | 0.03 | 0.03 |
| aloe vera | 0.1 | 0.1 | 0.1 |
| chamomile extract | 0.1 | 0.1 | 0.1 |
| sodium hydroxide solution (10%) | 0.1 | 0.1 | 0.1 |
| d.i. water | 26.07 | 26.57 | 22.95 |
| pH | 5.22 | 5-6 | 5.15 |
| viscosity (cP) | 18000 | — | — |

The identity of the individual constituents used are described more fully on Table 2.

TABLE 2

| | |
|---|---|
| ethanol (95%) | supplied as SDA Alcohol 190 proof |
| Methocel ® E4M | hydroxypropylmethylcellulose supplied as |
| | Methocel ® E4M (100% wt. actives) (ex. Dow Chem. |
| | Co.) |
| glycerine | USP glycerine (100% wt. actives) |
| Opulyn ® | Opulyn 303B, opacifier preparation based on a |
| 303B (40%) | styrene/acrylamide copolymer emulsion (40% wt. |
| | actives), used as supplied (ex. Rohm & Haas Inc.) |
| Cosmedia ® | Cosmedia Triple C, technical mixture containing |
| Triple C | cationic Polyquaternium-type polymer, emollient, |
| (90-100%) | and surfactant, optionally further containing water, |
| | used as supplied (ex. Cognis) |
| Cetiol ® Sensoft | Cetiol Sensoft, propylheptyl caprylate, an emollient, |
| | used as supplied (ex. Cognis) |
| Polyox ® | Polyox ® WSR 205, powder composition |
| WSR 205 | comprising at least 95% wt. of poly(ethylene oxide) |
| | (14,000 molecular weight) polymer, 0-3% wt. |
| | fumed silica, and 0-1% wt. of mixed calcium salts |
| | (ex. Dow Chem. Co.) |
| SiClone ® SR-5 | SiClone ® SR-5, technical mixture of C13-C16 |
| | isoparaffins, C12-C14 isoparaffins, and C13-C15 |
| | alkane(s) (ex. Presperse LLC) |
| DC 200 fluid | dimethicone containing liquid composition |
| | composition, used as supplied, (ex. Dow Corp.) |
| fragrance | proprietary composition of its supplier |
| aloe vera | aloe vera, used as supplied (100% wt. actives) |
| | (laboratory grade) |
| chamomile | chamomile extract, used as supplied (100% wt. |
| extract | actives) (laboratory grade) |
| sodium hydroxide | aqueous sodium hydroxide solution (10% actives) |
| solution (10%) | |
| d.i. water | deionized water |

The viscosity oldie compositions C1, E1-E4 of Table 1 were evaluated utilizing a Brookfield Type RVT viscometer using a standard #6 spindle, rotating at 6 rpm, with the tested sample at room temperature (20° C.).

The topical germicidal compositions of the invention described on Table 1, were produced according to the following general protocol, all constituents were at room temperature (approx. 20-22° C.);

A first premix was formed by combining the cellulose constituent and the glycerin in a laboratory beaker with a spatula to ensure that the hydroxymethylcellulose was well mixed and wetter, and that a slurry was formed. Next, a mixture was made by adding into a lame laboratory beaker was added the water and the opacifier constituent which were blended together using a motorized laboratory mixer equipped with propeller, and mixing continued until the composition was homogenous. Thereafter to the large beaker was added the said first premix, and mixing continued until the composition was again homogenous. Next, about one-fifth of the ethanol was slowly added to the large beaker and mixing was continued until the composition was again homogenous, and thereafter was allowed to mix a further 30 minutes to ensure that the cellulose constituent was hilly swelled. Next, the remaining balance of the ethanol was slowly added, and mixing was continued until the composition was again homogenous. The motorized laboratory mixer equipped with the propeller as removed, and the propeller was substituted with a larger, U-shaped (or “anchor shaped”) mixing, impeller, which was inserted into the large laboratory beaker. This U-shaped mixing impeller was installed and used thereafter for all subsequent mixing, as its shape provided for more thorough mixing, including mixing near the vertical sidewalls of the laboratory beaker. The laboratory mixer was energized to run at approximately 400 rpm to ensure homogeneity of the mixture, after which the rpm were increased to about 1500-2000 rpm. Thereafter the Cosmedia® Triple C was slowly added under mixing conditions, and mixing continued a medium speed until the composition, in the large beaker was again homogenous. Thereafter, the following remaining constituents were individually sequentially added, under constant mixing to ensure that each added constituent was fully blended and the composition in the large beaker was again homogenous prior to the addition of the next of the said remaining constituents. Finally, the pH was checked, and when needed a measured amount of the aqueous sodium hydroxide solution (10% actives) was added under mixing conditions to adjust the pH of the composition to the target pH, as indicated on Table 1. The composition was then removed from the large beaker, and was ready for use or alternately could be stored in an appropriate storage container prior to or between uses.

Certain of the foregoing compositions described within Table 1 were subjected to accelerated ageing testing at a variety of temperatures and at either ambient humidity or increased humidity levels (−10° C., 25° C., 30° C./75% relative humidity (“r.h.”), 40° C./75% relative humidity, 50° C., and 60° C.) for several weeks. Sample aliquots of tested samples were contained in screw capped glass sample jars. The samples were evaluated at certain intervals for appearance, viscosity and pH at each interval. Viscosity was evaluated utilizing a Brookfield Type RVT viscometer using a standard #6 spindle, rotating at 5 rpm, alter the samples were allowed to equilibrate by resting on a laboratory benchtop to a testing temperature of room temperature (20° C.). Any deviation the initial aethetics of the as-mixed samples were indicated, with “o.k.” indicating that the tested sample retained the original as-mixed appearance and tactile characteristics. Where the samples were not tested under certain times or conditions, such is indicated by “nt.”. The results of these evaluations are indicated on the following tables.

TABLE 3A
composition E2

| | | | | 30° C./ | 40° C./ | | |
|---|---|---|---|---|---|---|---|
| | −10° C. | 4° C. | 25° C. | 75% r.h. | 75% r.h. | 50° C. | 60° C. |
| 1 week | | | | | | | |
| aesthetics | o.k. | o.k. | o.k. | o.k. | o.k. | o.k. | o.k. |
| viscosity (cP) | 34800 | 28000 | 26000 | 28000 | 30000 | 28000 | 28000 |
| pH | 5.44 | 5.4 | 5.53 | 5.53 | 5.63 | 5.77 | 5.97 |
| 4 weeks | | | | | | | |
| aesthetics | n.t. | o.k. | o.k. | o.k. | o.k. | o.k. | n.t. |
| viscosity (cP) | n.t. | 30000 | 26000 | 29000 | 32000 | 32000 | n.t. |
| pH | n.t. | 5.31 | 5.44 | 5.57 | 5.65 | 5.74 | n.t. |

TABLE 3B
compostion E5

| | | | | 30° C./ | 40° C./ | | |
|---|---|---|---|---|---|---|---|
| | −10° C. | 4° C. | 25° C. | 75% r.h. | 75% r.h. | 50° C. | 60° C. |
| 1 week | | | | | | | |
| aesthetics | o.k. | o.k. | o.k. | o.k. | o.k. | o.k. | o.k. |
| viscosity (cP) | 24000 | 24000 | 24000 | 26000 | 25000 | 26000 | 26500 |
| pH | 5.44 | 5.40 | 5.53 | 5.53 | 5.62 | 5.77 | 5.97 |
| 4 weeks | | | | | | | |
| aesthetics | n.t. | o.k. | o.k. | o.k. | o.k. | o.k. | n.t. |
| viscosity (cP) | n.t. | 27000 | 25000 | 25000 | 28000 | 28000 | n.t. |
| pH | n.t. | 5.63 | 5.83 | 5.75 | 5.74 | 5.83 | n.t. |

TABLE 3C
composition E6

| | | | | 30° C./ | 40° C./ | | |
|---|---|---|---|---|---|---|---|
| 1 week | −10° C. | 4° C. | 25° C. | 75% r.h. | 75% r.h. | 50° C. | 60° C. |
| aesthetics | o.k. | o.k. | o.k. | o.k. | o.k. | o.k. | o.k. |
| viscosity (cP) | 20000 | 18000 | 18000 | 18000 | 18000 | 18000 | 24000 |
| pH | 5.88 | 5.85 | 5.7 | 5.8 | 5.9 | 5.97 | 5.75 |

TABLE 3D
composition E7

| | | | | 30° C./ | 40° C./ | | |
|---|---|---|---|---|---|---|---|
| | −10° C. | 4° C. | 25° C. | 75% r.h. | 75% r.h. | 50° C. | 60° C. |
| 1 week | | | | | | | |
| aesthetics | n.t. | n.t. | n.t. | n.t. | n.t. | n.t | o.k. |
| viscosity (cP) | n.t. | n.t. | n.t. | n.t. | n.t. | n.t. | 20000 |
| pH | n.t. | n.t. | n.t. | n.t. | n.t. | n.t. | 5.75 |
| 3 weeks | | | | | | | |
| aesthetics | n.t. | o.k. | o.k. | o.k. | o.k. | o.k. | n.t. |
| viscostity (cP) | n.t. | 22000 | 22000 | 22000 | 28000 | 24000 | n.t. |
| pH | n.t. | 5 | 5 | 5.1 | 5.4 | 5.48 | n.t. |

The sample E7 was also subjected to a “freeze/thaw” test wherein the sample was frozen, then thawed to mom temperature, then tested. Following this test the appearance was unchanged (“o.k.”), the viscosity was 23000 and the PH was 5.1.

TABLE 3E
composition E8

| | | | | 30° C./ | 40° C./ | | |
|---|---|---|---|---|---|---|---|
| 3 weeks | −10° C. | 4° C. | 25° C. | 75% r.h. | 75% r.h. | 50° C. | 60° C. |
| aesthetics | n.t. | o.k. | o.k. | o.k. | n.t. | o.k. | n.t. |
| viscostity (cP) | n.t. | 16000 | 16000 | 16000 | n.t. | 17000 | n.t. |
| pH | n.t. | 5.52 | 5.5 | 5.6 | n.t. | 5.63 | n.t. |

TABLE 3F
composition E9

| | | | | 30° C./ | 40° C./ | | |
|---|---|---|---|---|---|---|---|
| 3 weeks | −10° C. | 4° C. | 25° C. | 75% r.h. | 75% r.h. | 50° C. | 60° C. |
| aesthetics | n.t. | o.k. | o.k. | o.k. | o.k. | o.k. | n.t. |
| viscostity (cP) | n.t. | 24000 | 22000 | 20000 | 32000 | 28000 | n.t. |
| pH | n.t. | 5.53 | 5.7 | 5.5 | 5.7 | 5.8 | n.t. |

TABLE 3G
composition E11

| | | | | 30° C./ | 40° C./ | | |
|---|---|---|---|---|---|---|---|
| 1 week | −10° C. | 4° C. | 25° C. | 75% r.h. | 75% r.h. | 50° C. | 60° C. |
| aesthetics | o.k. | o.k. | o.k. | o.k. | o.k. | o.k. | o.k. |
| viscostity (cP) | 20000 | 18000 | 20000 | 20000 | 22000 | 20000 | 24000 |
| pH | 5.92 | 5.84 | 5.9 | 6.0 | 6.0 | 6.05 | 6.06 |

The sample E11 was also subjected to a “freeze/thaw” test wherein the sample was frozen, then thawed to room temperature, then tested. Following this test the appearance was unchanged (“o.k.”), the viscosity was 23000 and the pH was 5.9.

As evident from the foregoing formulations and results, the compositions exhibited a surprising degree of viscosity increase even at low levels, viz., not more than 0.25% wt, or even not more than 0.1% wt. of the film forming constituent based on one or more celluloses or cellulose derivatives, and excellent retention of the initial viscosity and appearance over various storage testing regimens.

1. A topical germicidal composition comprising:
50-85% wt. of an alcohol constituent comprising one or more C₁-C₄ monohydric alcohols; 0.01-5% wt. of a film forming constituent based on one or more celluloses or cellulose derivatives;
0.01-25% wt. of a humectant;
0.01-5% wt. of an opacifier constituent; and
with the balance of the composition, comprising water;
wherein the composition is flowable and;
wherein subsequent to being stored at elevated temperatures and/or extended time intervals, is retained as a single phase composition and does not split or separate into two or more phases.

2. Topical germicidal composition according to claim 1 further comprising a Polyquaternium-type polymer or material.

3. Topical germicidal composition according to claim 1 comprising 55-70% wt. of the alcohol constituent.

4. Topical germicidal composition according to claim 1 comprising 0.01-1% wt. of the film forming constituent.

5. Topical germicidal composition according to claim 1 further comprising hydroxypropylmethylcellulose.

6. Topical germicidal composition according to claim 1 comprising not more than 0.25% wt. of the film forming.

7. Topical germicidal composition according to claim 1 comprising not more than 0.1% wt. of the film forming constituent.

8. An improved method for the treatment of a body surface comprising:
applying an effective amount of a topical germicidal composition according to claim 1 to the body surface, in order to provide an effective cleaning and/or germicidal benefit.

9. A method according to claim 8, wherein the topical germicidal composition is effective against one or more, of the following microorganisms: B. cepacia, E. coli, S. aureus, S. marcenscens, S. pyogenes, S. epidermidis, E. faecalis, K. pneumoniae, P. aeruginosa, E. hirae, S. pneumoniae, C. albicans, S. enterica, and methicillin resistant Staphylococcus aureus (“MRSA”).

10. Topical germicidal composition according to claim 1, wherein the humectants comprises glycerine.

11. Topical germicidal composition according to claim 1 further comprising one or more additional constituents for improving at least one technical characteristic of the composition.

12. Topical germicidal composition according to claim 1 comprising not more than about 30% wt. of water.

13. Topical germicidal composition according to claim 1, wherein the composition exhibits an initial viscosity (“as mixed”) of at least about 10 cPs measured at 25° C.

14. Topical germicidal composition according to claim 1, wherein the composition provides a topical germicidal benefit when applied to the skin or parts of the body.

15. Topical germicidal composition according to claim 11 comprising not more than about 25% wt. of the one or more additional constituents.

16. Topical germicidal composition according to claim 11, the one or more additional constituents selected from the group consisting of hydrophilic or lipophilic gelling agents, hydrophilic or lipophilic active agents, emulsifiers, particulates, fillers, emollients, skin conditioning agents, preservatives, antioxidants, organic or inorganic solvents, pH adjusting agents, pH buffers, chealating agents, fragrances, fillers, preservatives, dyestuffs or colorants, light stabilizers, and UV absorbers.
